(19) United States
(12) Patent Application Publication
(10) Pub. No.: US 2007/0190168 A1
(43) Pub. Date: Aug. 16, 2007

(54) Method of the treatment and prophylaxy of some diseases and rejuvenation of the organism

(76) Inventor: Yakov Yevseyevich Brilman, Voorhees, NJ (US)

Correspondence Address: FLASTER/GREENBERG P.C.;8 PENN CENTER, 1628 JOHN F. KENNEDY BLVD., 15TH FLOOR, PHILADELPHIA, PA, 19103, US

(21) Appl. No.: 11/335,779

(22) Filed: Jan. 20, 2006

(51) Int. Cl. A61K 35/22

(52) U.S. Cl. 424/545

(57) ABSTRACT

The invention is intended for treatment and prophylaxy of infectious diseases and others in which urine from a sick person contains antigens or gaptens from the incitant of the disease (for reception autourine vaccine or urine vaccine). It is also exposed to necessary processing as well as for treatment of the diseases in accordance with presence of agressive antibodies (diabetes and others), and malignant new formations. Urine from a sick person, containing antigens from the incitant of the disease, cleaned from urates and subjected to necessary processing results in killing (inactive) and live weakened vaccine which can be successfully used as an inoculative of the material for prophylaxy of this disease. Also, it is offered to use the processed urine from children and teenagers, at the point when there are the most contents of hormones in the organism. This includes growing hormones, sexual hormones and others needed for achievement of the effect of the rejuvenation of the recipient. The processed urine is entered into the recipients organism with parenteral injections.

### BACKGROUND OF THE INVENTION

1. The invention pertains to the area of medicine and is intended for the treatment and prevention of the infections diseases (viral hepatitis, HIV/AIDS disease and other) and also diseases in accordance with the presence of aggressive antibodies (diabetes and others), malignant new formations and others.

For treatment of sick person this method uses urine from the sick person which is subject to necessary processing: the liberation of the urine from urates, bring the PH to 7, 4 boiling 1-2 min. in one variant of the use and long heating under 56 degrees C 30-40 min. followed by ultraviolet radiation and is entered with injections in the organism of the person with adjuvant, but under malignant new formations—in combination with 1% solution of methylen blue and 33% solution of the ethyl alcohol.

Urine from a sick person, containing antigens of the incitant of the disease, cleaned from urates and subjected to necessary processing as a result of which are formed a killing (inactive) and living weakened vaccines, can be successfully used as inoculative of the material for warning of this disease.

In order to achieve certain rejuvenating effect, it is necessary to use the adjuvant urine or a newborn and children under 1-2 years as well as urine from teenagers (13-19 years), which contains, in the blood and in the urine, hormones of the growing, sexual and others in the greatest amount.

Herewith, all variant injections of the proposed urine (with the exclusion) of intravenous) with adjuvant are used in the organism of the recipient on schedule.

2. Description of Related Art

As known in popular medicine the urine of a sick person is used for the treatment of some diseases. In some cases the sick person is offered to drink this urine. This has a weak medical effect.

Urine is a biological liquid, worked over by the kidneys, and a result of ultrafiltration of the plasma blood in the kidneys.

Urine is a valuable as blood in laboratory diagnostics of most diseases. In urine, it is possible to fine toxins, antigens of the causes of the diseases, as well as aggressive autoantibodies, which can damage the persons own organs and system tissues.

Urine can be valuable stimulant factor of the immunizations of the organism against aggressive autoantibodies in diabetes and other similar diseases and can suppress the pathological influence of these agents.

The antigens and toxins the urine contains can stimulate the immune system of the sick person and can be used as vaccines for preventing some disease.

This can be reached by the influence of the high temperature or long heating of the urine. It may produce the stereochemical alteration in the molecular construction of urine contents.

Introduction to the organism brings about additional influences on the immune system, intensifying it in one event (at immunodeficiency), in particular, and creating additional reactivity in the other (oppressing the influence of aggressive autoantibody in diabetes and other). Thus, it follows, that urine from a sick person can be used as effectively as blood is used in medical practice corresponding to its use after necessary processing and proposed method of the introduction to the organism for necessary reception of immune reactions. There is material available for making an autovaccine or vaccine, containing antigens infection.

The proposed method is different from the prototype in the following:

1) The urine from the sick person is injected into an organism material.

2) It is used only on the sedimentary part of the urine.

3) The urine is used with an adjuvant.

4) The urinary mixture is subjected to thermal influence or ultra-violet irradiation.

5) Bring the PH from urine to the PH blood (b 7,4).

6) Other chemical preparations are added to the urine depending on disease.

For the efficient treatment and prophylaxy of the many diseases (diabetes and other autoimmune diseases, malignant new formation, viral hepatitis, HIV/AIDS diseases, grippe and others) by means of use of urine from a sick person the following is offered: use it in two variants—1) In condition of observance of the strict aseptic, take 20-25ml. of the over sedimentary part liquid urine (after 1-2hours of the elimination for fall of the salts of the urines acids or using other methods to achieve the same results) and add to it as an adjuvant 5 ml. of the same over sedimentary part of a liquid urine from another sick person suffering from such diseases, but excluding the possibility of the contamination of the disease. It is possible, using another adjuvant.

Then, define the following PH mixture: if urine has an acid reaction that brings its PH to 7,4 (PH blood), add to it a sterile 10solution of bicarbonate soda. However if the mixture is alkaline, then acidify it with sterile 10solutions of ascorbinic acid and also bring to such PH. After this urinary mixture warms up, before boiling and use for injections, intradermal, subdermal and intramuscular. For the following procedure, warm up the prepared urinary mixture to 56 degrees C for 30 min. and enter it parenteral as under the first procedure.

The liquid from the sediment of urine from the sick person, as it is described above, without adjuvant, i.e. boiled urine of sick person and keep the urine warmed at 56 degrees C for 30 min. are injected into this person alternately.

For the treatment of a sick person with malignancies, it is offered to use the combination of autohemo-autovaccino-immunotherapy and autourinotherapy, i.e. intramuscular, intradermal and hypodermal injections introducing consequentially boiled urine from the sick person and keeping the urine warmed at 56 degrees C for 30 min. as it is described above, with injection of a 1% solution of methylene blue(used in medicine as an antidote for some poisoning) for intravenous of 50 ml. or more, alternatively with intravenous introduction of 30% solution alcohol ethyl. Herewith consider the absence of the cellular wall beside malignant cells that will promote greater absorption and fixing of the introduced paint from these cells and also in the possible absence or weak participating malignant cells in the oxygenated-reconstruction processes the methylene blue, that can promote destruction of these cells (this needs experimental confirmation).

Scheme of the Treatment:

By the first procedure the sick person receives injections using the mixture of urines on first variant alternately intradermal in the skin of the internal surface of the forearm 0.1-0.2 ml., subdermal in the place of the scapula −3 ml., and intramuscular (in buttocs) −5 ml., Apply injections the following day of the mixture “A”) (according to description above mentioned of the invention).

On the third day, conduct injections (second procedure of this method using the mixture of the urine on first variant also as it is described for the first day. After procedures alternate in one day with mixture “B” (according to same description above noted invention).

In the following procedure use in one day the prepared urine of the sick person on second variant as it is described above—enter intravenous 5-10ml., intramuscular 5 ml., subdermal −3 ml. and intradermal −0.1-0.2 ml. each increase the amount of the places injections on one and bring them before 10 injections.

For prophylaxis of some infectious diseases (grippe, HIV/AIDS and others) use the liquid over the sediment of urine from sick person with this infection, subjected to boiling for 1-2 minutes or long heating under 56 degrees C for 30-40 min. and more with the purpose of creating weakened or killed vaccines, which is entered into the organisms via intramuscular, intradermal and subdermal injections in the necessary volumes.

Use the alternative injections in the organism of killed vaccine and weakened alive vaccine. One and the same antigens reinforce the immune reactivity from the organism and that can be successfully used for treatment and prophylaxy in multiple diseases, under which there are antigens of the source of this disease in urine.

For treatment of certain effects of the rejuvenation the organism is offered to use alternately as adjuvant urine, subject to above described processing taken from newborns and children before 2-3 years. Such urine contains hormones of the growing (hormon hypophysis—somototropin) contributory process of the rejuvenating organism.

Together or alternately it is offered to introduce in the organism recipient processed urine from teenagers (13-19years) At this age urine has raised the contents of sexual hormones, biostimulate effect which is known and hormones from epiphys—melatonin (its high contents in blood exists after 8:00 pm in the evening, consequently and urine in this time also contains more). The rejuvenating efficiency of this hormone is also known. In this connection taking the urine from this contingent donor follows to conduct at an evening time apart from girls (for women) and from boys (for men).

Taking the urine must abide by of all rules of the asepsis with following ultraviolet irradiation for the reason of preventive disinfections and used in the manner of adjuvant in the mixture with the own urine of recipient in correlations: on four parts of over sedimentary urine from donor—1 part of over sedimentary boiled urine from recipient. With the exception of possible allergy of the organisms as a result of repeated introductions of one and same alien components urine from one and same donor, it is offered for each procedure to use urine from different sound donors.

The proposed composition enters the organism with subdermal and intramuscular injections in 3-5 ml. and more.

In addition, use the necessary physical exercised and massage of the body in the skin as proposed by me as biologically active material.

For more successful achievement of the effect of rejuvenating the organism use the following proposed method combined with the method of autohemo-autovaccine-immunotherapy (see patent No. . . ) with using the blood from same said donors.

Note:

Extract of the placenta can be used instead of the blood from children of early age for injection.

The contents of the urine includes not only material for diagnosing many diseases, but contains components which are successfully used in medical practice.

Aside from varied material that can be found in urine, it is possible to find antigens incitant of the disease and antibodies against them.

In urine, there are also various materials which are produced by the organism's hormones and ferments and participate in all of the biological processes of the organism.

All of this was taken into account in the proposed invention for their preventive material, after cleaning from urates and necessary additional heating; as well as other processes for receipt of autovaccines.

They can be used in medical practice against many diseases and vaccines for preventing infectious disease (HIV/AIDS infections, grippe, virus hepatitis, etc), as well as diseases in accordance with the presence of aggressive autoantibody (diabetes and etc), in the field of oncology, as well as for preventing the aging processes of the organism.

What is claimed is:

1. The method of the treatment and prophylaxy of some diseases using the urine from a sick person in combination with other methods.

2. The method of claim 1 wherein the urine from a sick person is used without urates and at necessary PH.

3. The method of claim 1 wherein it uses in addition the urine from sick person with the same disease as adjuvant.

4. The method of claim 1 wherein the autovaccine is the heated or boiled urine containing antigens from the infected person.

5. The method of claim 1 wherein the urine therapy is used in combination with autohemo-autovaccino-immunotherapy and with sol. methylenblau (under malignant new formation), 30% sol. alcohol ethyl and boiled own urine for intravenous injections.

6. The method of claim 1 wherein the urine from the sick person is heated under 56 degrees C 30 min. or more or boiled 1-2 min for use as a vaccine for prophylaxy of some infected diseases.

7. The method of claim 1 wherein the urine therapy and urinovaccine use intradermal, intramuscular, and subdermal injections on scheme.

8. The method of claim 1 wherein for rejuvenation the organisms is offered use of injection in the organism of the urine, after processing from new-born and children before 2-3 years, containing hormones of the growing and urine from sound teenagers (13-19 years) containing sexual and melatonin hormones.

9. The method of claim 8 wherein this urine from specified donors follows youngster to subject to ultraviolet irradiation for the reason of disinfection and to use as an adjuvant in mixture with boiled own urine recipient in necessary correlations.
